(19) United States
(12) Patent Application Publication
(10) Pub. No.: US 2009/0221838 A1
(43) Pub. Date: Sep. 3, 2009

(54) PROCESS FOR THE PREPARATION OF SUBSTITUTED PYRAZOLES

(76) Inventors: Meinrad Brenner, Steg (CH); Hans Williner, Glis (CH)

Correspondence Address: HOFFMANN & BARON, LLP, 6900 JERICHO TURNPIKE, SYOSSET, NY, 11791, US

(21) Appl. No.: 12/064,147

(22) PCT Filed: Aug. 18, 2006
(86) PCT No.: PCT/EP2006/008162

§ 371 Date: Jul. 1, 2008

(30) Foreign Application Priority Data
Aug. 18, 2005 (EP) 05017950.6

(51) Int. Cl. C07D 231/38

(52) U.S. Cl. 548/371.4

(57) ABSTRACT

The invention relates to an alternative process for the preparation of substituted 5-amino-pyrazoles, in which a carbonyl compound is reacted with a hydrazine derivative and cyanogen chloride to give an N-cyanohydrazone which is cyclized in the presence of a strong base.

The invention relates to an alternative process for the preparation of substituted 5-aminopyrazoles.

Compounds of the formula I,

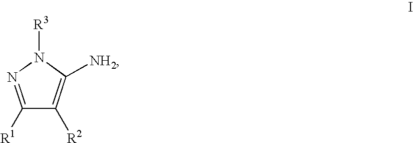

such as that in which R¹ is cyclopropyl, R² is hydrogen and R³ is methyl, are synthetic building blocks which can be used, for example, for the preparation of azetidines. According to WO-A-2003/077907 and WO-A-2005/026113, azetidines are suitable as CCR-3 receptor antagonists in the treatment of inflammation and allergic diseases.

With the well known process of Höhn, H., Z. Chem. 1970, 10, 386-388, cited in U.S. Pat. No. 3,894,005,5-aminopyrazoles of the formula I can be prepared from an acrylonitrile of the formula CHR¹═CR²CN, which is first reacted with hydrazine, an aldehyde or ketone to give the hydrazone and can subsequently be cyclized by treatment with sodium butoxide.

A process is known, from Ryckmans et al., Tetrahedron, 1997, 53, 1729-1734, in which activated enolizable ketones are reacted with hydrazines to give 1-cyano-2-vinyl-hydrazones, which are then converted, either thermally or in the presence of a base, to 4-substituted 5-aminopyrazoles of the formula I. The activating group R² in the 4-position of the pyrazole ring is preserved here. The activating group R² is preferably an acyl group. R=phenyl is already much less favoured. The cyclizing of a 5:1 E/Z-hydrazone mixture of the N-cyano-N-methylhydrazone of benzyl methyl ketone (R²=phenyl) results in 1,3-dimethyl-4-phenyl-5-aminopyrazole in a yield of 66%. The formation of 1,4-dimethyl-3-phenyl-5-aminopyrazole is not reported. Without an activating group, thus, for example, with R²=hydrogen or C₁₋₆-alkyl, it was not possible to obtain 5-aminopyrazole according to the Ryckman process.

Other processes for the preparation of 5-amino-1,3-dimethylpyrazole are disclosed in WO-A-94/13661 and WO-A-95/34563.

It was an object of the invention to make available an alternative process for the preparation of substituted 5-aminopyrazoles. To improve the economics of the process, the substituents R¹ to R³ should in addition already be able to be introduced into the molecule in the synthesis of the ring in order to avoid later substitution.

This object is achieved according to Claim 1.

A process is claimed for the preparation of substituted pyrazoles of the formula

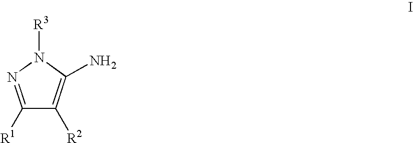

in which R¹ is chosen from the group consisting of hydrogen, C₁₋₆-alkyl, C₁₋₆-alkoxy, C₃₋₆-cycloalkyl, aryl and heteroaryl, in which, apart from hydrogen, each R¹ substituent can, if appropriate, carry one or more substituents from the group consisting of C₁₋₆-alkyl, C₁₋₆-alkoxy, halogen and nitro, and
R² is chosen from the group consisting of hydrogen, cyano, halogen, C₁₋₆-alkyl, C₁₋₆-alkoxy, C₁₋₆-alkoxycarbonyl, C₃₋₆-cycloalkyl, aryl and heteroaryl, in which, apart from hydrogen, cyano and halogen, each R² substituent can, if appropriate, carry one or more substituents from the group consisting of C₁₋₆-alkyl, C₁₋₆-alkoxy, halogen and nitro, or in which R¹ and R² together represent a —(CH₂)ₙ— group where n=3, 4 or 5 which can, if appropriate, comprise one or more halogen atoms, and
in which R³ is chosen from the group consisting of C₁₋₆-alkyl, C₃₋₆-cycloalkyl, aryl and heteroaryl, in which each R³ substituent is, if appropriate, substituted with one or more halogen atoms,
by reacting, in a first stage, a compound of the formula

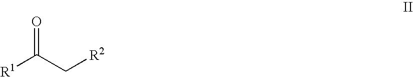

in which R¹ and R² are as defined above,
with a compound of the formula

NH₂NHR³ III

in which R³ is as defined above,
to give a compound of the formula

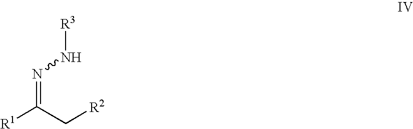

in which R¹, R² and R³ are as defined above,
which then, in a second stage, is reacted with cyanogen chloride in the presence of a base to give a compound of the formula

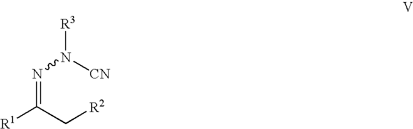

in which R¹, R² and R³ are as defined above,
which, in the final stage, is converted in the presence of a strong base to give a compound of the formula I.

The compounds of the formula II can be aldehydes or ketones. In the case of asymmetric ketones, two different compounds of the formula I may be formed. The product which predominates depends on the steric and electronic properties of the substituents R¹ and R² and also on the reaction conditions. However, the two products always differ appreciably from one another, so that they can be easily separated. If R¹ and R² together represent a —(CH₂)ₙ— group with n=3, 4 or 5, the compound of the formula II is a cyclic ketone. Examples of cyclic ketones of the formula II are cyclopentanone, cyclohexanone or cycloheptanone.

In a preferred process, the compound of the formula II is chosen from the corresponding column in Table 1. The “Compound of the formula I” column each time gives the compound predominantly formed in the reaction. The R³ radical in the molecule results from the hydrazine derivative of the formula III used each time.

TABLE 1

| | Compound of the | Compound of the |
|---|---|---|
| | formula II | formula I |
| a) | 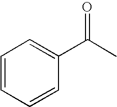 | 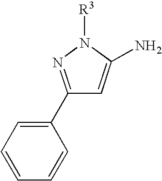 |
| b) | 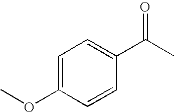 | 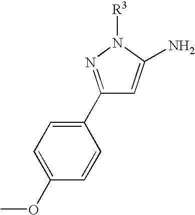 |
| c) | 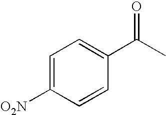 | 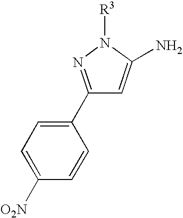 |
| d) | 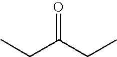 | 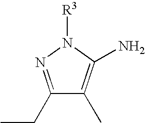 |
| e) | 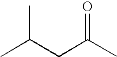 | 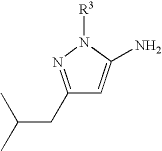 |
| f) | 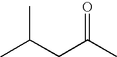 | 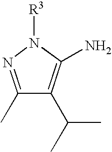 |
| g) | 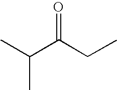 | 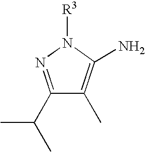 |
| h) | 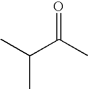 | 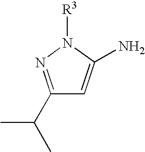 |
| i) | 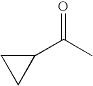 | 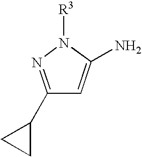 |
| j) | 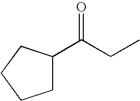 | 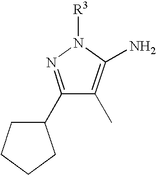 |
| k) | 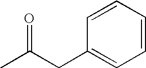 | 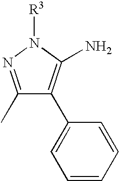 |
| l) | 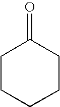 | 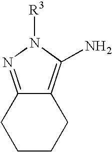 |
| m) | 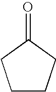 | 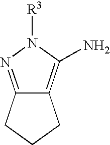 |
| n) | 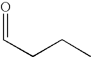 | 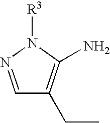 |
| o) | 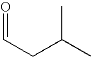 | 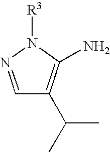 |

Additional examples of preferred process variants, in which the hydrazine derivative of the formula III used is defined and accordingly the R³ radical is defined, are given in Table 2.

TABLE 2

| | Compound | Compound | Compound |
|---|---|---|---|
| | of the | of the | of the |
| | formula II | formula III | formula I |
| a) | 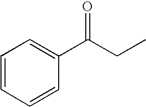 | NH₂NHCH₃ | 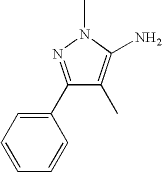 |
| b) | 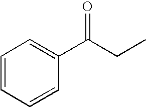 | 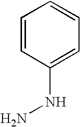 | 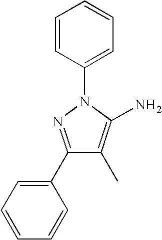 |
| c) | 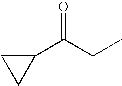 | 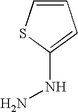 | 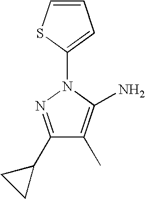 |

In a particularly preferred process variant, R¹ is cyclopropyl, R² is hydrogen and R³ is methyl.

Here and subsequently, the expression “C₁₋ₙ-alkyl” denotes an unbranched or branched alkyl group with 1 to n carbon atoms. Thus, C₁₋₇-alkyl represents, for example, groups such as methyl, ethyl, propyl, isopropyl, butyl, isobutyl, sec-butyl, tert-butyl, pentyl, hexyl, heptyl or 1,4-dimethylpentyl.

Here and subsequently, the expression “C₁₋ₙ-alkoxy” denotes an unbranched or branched alkoxy group with 1 to n carbon atoms. Thus, C₁₋₇-alkoxy represents, for example, groups such as methoxy, ethoxy, propoxy, isopropoxy, butoxy, isobutoxy, sec-butoxy, tert-butoxy, pentyloxy, hexyloxy, heptyloxy or 1,4-dimethylpentyloxy.

Here and subsequently, the expression “C₃₋₆-cycloalkyl” denotes a cycloalkyl group with 3 to 6 carbon atoms and represents cyclopropyl, cyclobutyl, cyclopentyl and cyclohexyl.

Here and subsequently, the term “aryl” is understood to mean in particular an aromatic group with 6 to 10 carbon atoms, such as, for example, phenyl, p-tolyl or naphthyl.

Here and subsequently, the term “aralkyl” is understood in particular to mean an alkyl group substituted with an aryl group, such as, for example, phenylethyl, the alkyl group comprising from 1 to 4 carbon atoms and the aryl group comprising from 6 to 10 carbon atoms, as defined above.

Here and subsequently, the term “heteroaryl” is understood to mean in particular a heteroaromatic group with 4 to 8 carbon atoms, such as, for example, 2- or 3-furanyl, 2- or 3-thiophenyl or 2-, 3- or 4-pyridinyl.

Here and subsequently, the expression “halogen” denotes fluorine, chlorine, bromine or iodine.

In a preferred process variant, the base used in the second stage is an inorganic base, preferably chosen from the group consisting of alkali metal and alkaline earth metal hydroxides, alkali metal and alkaline earth metal carbonates, trisodium phosphate and mixtures thereof.

The first stage is preferably carried out at the reflux temperature of the chosen solvent. The progress of the reaction can be determined very easily by thin layer chromatography or gas chromatography.

The product from the first stage does not have to be isolated and can be directly further reacted.

In the second stage, the hydrazine derivative formed in the first stage is reacted with cyanogen chloride in the presence of a base. Inorganic bases are especially suitable for the second stage and can be chosen from the group consisting of alkali metal and alkaline earth metal hydroxides, alkali metal and alkaline earth metal carbonates, trisodium phosphate and mixtures thereof. Use is particularly preferably made, as base, of an alkali metal carbonate and in this connection particularly of potassium carbonate.

In a preferred process variant, the cyanogen chloride is used in the second stage as a gas or dissolved in a solvent. In the process according to the invention, it does not matter whether the reaction mixture from the first stage is added to the cyanogen chloride or the cyanogen chloride is added to the reaction mixture.

In a particularly preferred process variant, the second stage is carried out at a temperature between −100 and 0° C., particularly preferably between −70 and −20° C.

Since the product from the first stage can be directly further reacted with cyanogen chloride in the second stage, it is particularly advantageous to carry out the reactions of the first and second stages as a “one pot reaction”.

In particular, solvents for the first and second stages can be chosen from the group consisting of cyclohexane, hexane, heptane, petroleum ether, ethanol, diethyl ether, methyl tert-butyl ether (MTBE), tetrahydrofuran (THF), toluene, xylene and mixtures thereof. MTBE, THF and toluene are particularly preferred.

The term “petroleum ether” is to be understood as meaning generally industrial alkane mixtures with a relatively broad boiling point range, but also in particular mixtures of isomers, for example of hexane and heptane.

The strong base used in the third stage must be able to deprotonate the carbon atom bonded directly to the R² group. Preferably, the strong base is chosen from the group consisting of metal hydrides, metal amides, metal alkoxides and organometallic compounds. NaH or KH is preferably used as metal hydride. Metal amides are preferably chosen from the group consisting of sodium amide, lithium diisopropylamide (LDA) and the lithium amide of tetramethylpiperidine (Li-TMP). Use is preferably made, as metal alkoxides, of sodium ethoxide and potassium tert-butoxide. Organometallic compounds are preferably chosen from the group consisting of n-butyllithium, sec-butyllithium and tert-butyllithium.

In a particularly preferred process variant, the base is chosen from the group consisting of lithium diisopropylamide, potassium tert-butoxide, n-butyllithium, sec-butyllithium and tert-butyllithium.

In a particularly preferred process variant, the third stage is carried out at a temperature of between −100 and 0° C., particularly preferably between −70 and −20° C.

For the third stage, the solvent has to be inert with regard to the strong base used. In Example 3, a change in solvent is carried out but this is not mandatory. In a preferred process variant, no change in solvent takes place between the second and third stages.

A solvent which is inert to all the reagents of the three stages can be used in order to carry out the reactions of the first, second and third stages as a “one-pot reaction”.

For the third stage, the solvent is preferably chosen from the group consisting of cyclo-hexane, hexane, heptane, petroleum ether, diethyl ether, MTBE, THF, toluene, xylene and mixtures thereof. MTBE, THF and toluene are particularly preferred.

In the additional preferred process variant of a “one-pot reaction” comprising all three stages, the solvent is chosen from the group consisting of cyclohexane, hexane, heptane, diethyl ether, MTBE, THF, toluene, xylene and mixtures thereof, particularly preferably from MTBE, THF and toluene.

After the end of the reaction, the strong base is quenched, for example by addition of water, and the product can be isolated. Preferably, with solvents at least partially miscible with water, a salt, such as, for example, ammonium chloride, is added for phase separation.

### EXAMPLES

### Preparation of a Lithium Diisopropylamide Solution (LDA Solution)

1.6M n-butyllithium solution (108 ml, 173 mmol) in hexane (BuLi) was added at −60° C. to a mixture of diisopropylamine (19.0 g, 189 mmol) in 200 ml of THF and the mixture was stirred for 1 h. The LDA solution obtained was used directly in Example 3. Alternatively, however, a commercially available LDA solution or solid LDA can also be used, this being available, for example, from Fluka. If appropriate, solid LDA can be dissolved or suspended in a suitable solvent, for example in THF, MTBE or hexane, before use.

### Example 1

### (E/Z)-N-(1-Cyclopropylethylidene)-N′-methylhydrazine (mixture)

A mixture of cyclopropyl methyl ketone (12.6 g, 150 mmol) and methylhydrazine (11.0 g, 240 mmol) was heated with stirring in 100 ml of toluene at a temperature of 93° C. under reflux for 11 h. After the end of the reaction, the reaction mixture was cooled to 0° C. An (E/Z)-N-(1-cyclopropylethylidene)-N′-methylhydrazine mixture with an E/Z distribution of approximately 3:1 was obtained, from which an aliquot was removed and purified for the characterization.

¹H NMR (CDCl₃): δ=4.40 (br, 1H), 3.92 (s, 3H_{Z}), 3.90 (s, 3H_{E}), 1.72 (s, 3H_{Z}), 1.58 (m, 1H_{E}), 1.52 (s, 3H_{E}), 1.50 (m, 1H_{Z}), 0.80 (dt, 2H_{Z}), 0.70 (m, 2H_{Z}), 0.68 (m, 2H_{E}), 0.62 ppm (m, 2H_{E}).

### Example 2

### (E/Z)-N-Cyano-N′-(1-cyclopropylethylidene)-N-methylhydrazine (mixture)

The bulk of the reaction mixture from Example 1 was, after cooling down, treated with an aqueous K₂CO₃ solution (27.6 g, 200 mmol, in 55 ml of water). Cyanogen chloride (14.0 g, 230 mmol) was introduced into this mixture at 0° C. over 90 min. The mixture was subsequently stirred at 0° C. for a further 2 h. After the end of the reaction, the organic phase was separated off and toluene was evaporated. The oily residue (22.8 g of crude product) was taken up in 100 ml of tetrahydrofuran (THF). An (E/Z)-N-cyano-N′-(1-cyclo-propylethylidene)-N-methylhydrazine mixture with an E/Z distribution of approximately 3:1 was obtained, from which an aliquot was removed and purified for the characterization.

¹H NMR (CDCl₃): δ=3.18 (s, 3H_{Z}), 3.16 (s, 3H_{E}), 2.18 (m, 1H_{Z}), 1.92 (s, 3H_{E}), 1.70 (s, 3H_{Z}), 1.68 (m, 1H_{E}), 1.80 (dt, 2H_{Z}), 0.86 (m, 2H_{Z}), 0.82 ppm (m, 4H_{E}).

### Example 3

### 5-Cyclopropyl-2-methyl-2H-pyrazol-3-ylamine

Approximately 300 ml of a freshly prepared approximately 0.6M LDA solution (see above) were treated, at −60 to −65° C., within 1 h with 113 g of the crude product solution from Example 2. Monitoring by thin layer chromatography resulted in complete conversion after 1 h.

After the end of the reaction, the reaction mixture was able to warm up to −10° C. and was then treated with a saturated NH₄Cl solution (30 ml). After separation of the phases, the organic phase was separated off and the aqueous phase was again extracted with THF (2×20 ml). The combined organic phases were dried over MgSO₄ and evaporated to dryness. The crude product (18.9 g) was obtained as a light-yellow solid with a yield of 92%, based on the original amount of cyclopropylethanone in Example 1.

¹H NMR (CDCl₃): δ=5.20 (s, 1H), 3.58 (s, 3H), 3.45 (br, 2H), 1.80 (m, 1H), 0.82 (m, 2H), 0.62 ppm (m, 2H).

### Example 4

### Recrystallization of 5-cyclopropyl-2-methyl-2H-pyrazol-3-ylamine

18.9 g of the product from Example 2 were dissolved at 65° C. in a mixture of diisopropyl ether (35 ml) and ethyl acetate (70 ml). Hexane (25 ml) was subsequently added and the temperature was slowly reduced to 10° C. The precipitated solid was filtered off and the product remaining in the mother liquor was once again recrystallized. Altogether, 13.4 g of 5-cyclopropyl-2-methyl-2H-pyrazol-3-ylamine (65% with regard to cyclopropylethanone) were isolated as a light-yellow solid.

1. Process for the preparation of substituted 5-aminopyrazoles of the formula 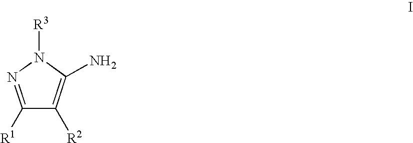
in which R¹ is chosen from the group consisting of hydrogen, C₁₋₆-alkyl, C₁₋₆-alkoxy, C₃₋₆-cycloalkyl, aryl and heteroaryl, in which, apart from hydrogen, each R¹ substituent can, if appropriate, carry one or more substituents from the group consisting of C₁₋₆-alkyl, C₁₋₆-alkoxy, halogen and nitro, and
R² is chosen from the group consisting of hydrogen, cyano, halogen, C₁₋₆-alkyl, C₁₋₆-alkoxy, C₁₋₆-alkoxycarbonyl, C₃₋₆-cycloalkyl, aryl and heteroaryl, in which, apart from hydrogen, cyano and halogen, each R² substituent can, if appropriate, carry one or more substituents from the group consisting of C₁₋₆-alkyl, C₁₋₆-alkoxy, halogen and nitro, or
in which R¹ and R² together represent a —(CH₂)ₙ— group where n=3, 4 or 5 which can, if appropriate, comprise one or more halogen atoms, and
in which R³ is chosen from the group consisting of C₁₋₆-alkyl, C₃₋₆-cycloalkyl, aryl and heteroaryl, in which each R³ substituent is, if appropriate, substituted with one or more halogen atoms,
by reacting, in a first stage, a compound of the formula 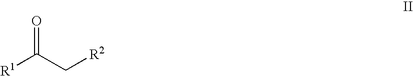
in which R¹ and R² are as defined above,
with a compound of the formula
NH₂NHR³ III
in which R³ is as defined above,
to give a compound of the formula 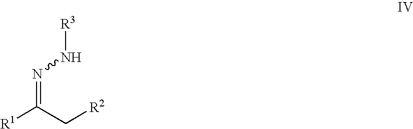
in which R¹, R² and R³ are as defined above,
which then, in a second stage, is reacted with cyanogen chloride in the presence of a base to give a compound of the formula 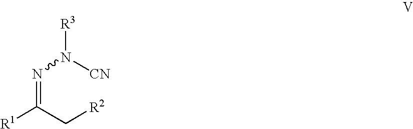
in which R¹, R² and R³ are as defined above,
which, in the final stage, is converted in the presence of a strong base to give a compound of the formula I.

2. Process according to claim 1, characterized in that the base used in the second stage is an inorganic base preferably chosen from the group consisting of alkali metal and alkaline earth metal hydroxides, alkali metal and alkaline earth metal carbonates, trisodium phosphate and mixtures thereof.

3. Process according to claim 1, characterized in that the reactions of the first and second stages are carried out as a “one-pot reaction”.

4. Process according to claim 1, characterized in that the strong base used in the third stage is chosen from the group consisting of metal hydrides, metal amides, metal alkoxides and organometallic compounds.

5. Process according to claim 4, characterized in that the strong base is chosen from the group consisting of lithium diisopropylamide, potassium tert-butoxide, n-butyllithium, sec-butyllithium and tert-butyllithium.

6. Process according to claim 1, characterized in that no change in solvent takes place between the second and third stages.

7. Process according to claim 5, characterized in that the reactions of the first, second and third stages are carried out as a “one-pot reaction”.

8. Process according to claim 7, characterized in that the solvent is chosen from the group consisting of cyclohexane, hexane, heptane, petroleum ether, diethyl ether, methyl tert-butyl ether (MTBE), tetrahydrofuran (THF), toluene, xylene and mixtures thereof.
